(19) United States
(12) Patent Application Publication
(10) Pub. No.: US 2014/0135821 A1
(43) Pub. Date: May 15, 2014

(54) SURGICAL NEEDLE LOCKS THAT COOPERATE WITH LAPAROSCOPIC FORCEPS AND RELATED METHODS

(71) Applicant: Wake Forest University Health Sciences, Winston-Salem, NC (US)

(72) Inventor: Rashann Coleman, Winston-Salem, NC (US)

(21) Appl. No.: 14/076,678

(22) Filed: Nov. 11, 2013

Related U.S. Application Data
(60) Provisional application No. 61/725,273, filed on Nov. 12, 2012.

(51) Int. Cl. A61B 17/29; A61B 17/34

(52) U.S. Cl. CPC A61B 17/29; A61B 17/3415
USPC 606/208

(57) ABSTRACT

Surgical laparoscopic forceps include slidably actuatable needle lock to lock jaws of the forceps closed while holding a suture needle trapped in the jaws of the forceps. The needle lock can be a band, ring, clip or sleeve that can be selectively deployed and is sized and configured to fit in a trocar over a shaft of the forceps.

### RELATED APPLICATIONS

This application claims the benefit of and priority to U.S. Provisional Application Ser. No. 61/725,273, filed Nov. 12, 2012, the contents of which are hereby incorporated by reference as if recited in full herein.

### FIELD OF THE INVENTION

The present invention relates to surgical tools for laparoscopic procedures.

### BACKGROUND

During some laparoscopic surgery procedures, laparoscopic forceps are used to grasp a suture needle so that a clinician can remove the forceps (holding the needle) through a trocar. Unfortunately, the forceps can emerge from the trocar outside the body without the needle (even though many forceps have locking mechanisms), which indicates that the needle was inadvertently dropped in the body somewhere. FIG. 1 shows a picture of a prior art laparoscopic forceps.

### SUMMARY OF EMBODIMENTS OF THE INVENTION

Embodiments of the present invention provide needle locks that cooperate with laparoscopic forceps to withdraw suture needles from a body during a laparoscopic procedure.

Surgical laparoscopic forceps include a needle lock that slidably receives the forcep jaws therein to lock jaws of the forceps closed while holding a suture needle trapped in the jaws of the forceps.

The needle lock can be a band, ring, clip or sleeve that can be selectively deployed and is sized and configured to fit in a trocar over a shaft of the forceps.

Embodiments of the invention are directed to laparoscopic surgical instruments. The instruments include: (a) forceps with a shaft attached to forcep jaws, the forceps being sized and configured to enter a trocar during a laparoscopic surgical procedure; and (b) a needle lock sized and configured to enter the trocar with the forceps and slidably receive at least a portion of the forcep jaws to clamp the forcep jaws shut while the forcep jaws hold a suture needle therebetween in a lock position.

The forcep jaws and needle lock can fit inside a trocar having a diameter between 3 mm and 20 mm, typically between 5-15 mm

The needle lock can have a maxima cross-sectional size of between 2-15 mm.

The forcep jaws can include cooperating first and second jaws with the first jaw having a slot therein and the second jaw having a padded surface thereon.

The forcep jaws can include projecting members and/or a surface treatment or material that cooperates with the needle lock to define a stop position for the needle lock.

The needle lock can be an elongate sleeve that abuts the shaft of the forceps.

The sleeve is selectively slidably advanced toward the forcep jaws from a pre-deployment position proximate a distal end of the trocar.

The sleeve can have a semi-rigid polymeric material and is sterile for medical use.

The needle lock can include a clip that slidably resides about an external surface of the shaft of the forceps.

The needle lock can include a ring or band that slidably resides about an external surface of the shaft of the forceps.

The surgical instrument can also include a pusher that is in communication with the needle lock and abuts the shaft of the forceps when inside the trocar.

The surgical instrument can include an external grip accessible by a user that is attached to a proximal end portion of the sleeve.

The surgical instrument can include a locking member that attaches to the shaft of the forceps to prevent rearward sliding of the needle lock once the needle lock is in the lock position over the forcep jaws.

Other embodiments are directed to methods of withdrawing a suture needle during a laparoscopic surgery. The methods include: (a) inserting forceps with a forceps shaft and forcep jaws with a needle lock through a trocar; (b) grasping a suture needle with the forcep jaws; then (c) slidably receiving at least a portion of the forcep jaws with the grasped suture needle into a needle lock to clamp the jaws shut while holding the suture needle; and then (d) withdrawing the grasped suture needle with the forcep jaws held inside the needle lock from the trocar.

The needle lock can include a sleeve. The slidably receiving step can be carried out by slidably advancing the sleeve a distance outside a distal end of the trocar toward the forcep jaws.

The needle lock can include a clip or ring. The slidably receiving step can be carried out by pushing the clip or ring a distance outside a distal end of the trocar toward the forcep jaws.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

Other systems and/or methods according to embodiments of the invention will be or become apparent to one with skill in the art upon review of the following drawings and detailed description. It is intended that all such additional systems, methods, and/or devices be included within this description, be within the scope of the present invention, and be protected by the accompanying claims.

### BRIEF DESCRIPTION OF THE DRAWINGS

Other features of the present invention will be more readily understood from the following detailed description of exemplary embodiments thereof when read in conjunction with the accompanying drawings.

FIG. 1 is a side perspective view of an exemplary prior art laparoscopic forceps.

FIGS. 2A and 2B are enlarged views of a distal end portion of forceps used to capture and hold a suture needle during a laparoscopic procedure.

FIG. 3A is a side view of a needle lock that cooperates with laparoscopic forceps according to embodiments of the present invention.

FIG. 3B is an enlarged view of a distal end portion of the device shown in FIG. 3A according to embodiments of the present invention.

FIGS. 4A-4C are side perspective sequential views of operation of the needle lock shown in FIG. 3A according to embodiments of the present invention.

FIG. 5 is a side perspective view of a surgical instrument assembly illustrating a needle lock and a locking mechanism to inhibit the needle lock from sliding off of the forcep jaws after it is in locking position according to embodiments of the present invention.

FIG. 6 is aside perspective view of forcep jaws with ridges or other position stop members according to embodiments of the present invention.

FIG. 7A is a side perspective view of a forceps with a sleeve needle lock according to embodiments of the present invention.

FIG. 7B are exemplary section views of two alternate designs taken along line 7B-7B in FIG. 7A.

FIG. 7C is a section view also taken along lines 7B-7B of FIG. 7A, illustrating an alternate forcep shaft configuration according to embodiments of the present invention.

FIG. 8 is a side view of an exemplary forceps shaft and a needle lock that fits over the shaft according to embodiments of the present invention.

FIGS. 9A-9C are sequential views of a different needle lock configuration according to embodiments of the present invention.

FIG. 10 is a flow chart of exemplary operations that can be used to lock suture needles inside forcep jaws for safe withdrawal out of a trocar according to embodiments of the present invention.

### DETAILED DESCRIPTION OF EMBODIMENTS OF THE INVENTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise. One or more features shown and discussed with respect to one embodiment may be included in another embodiment even if not explicitly described or shown with another embodiment.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The term “about” means that the recited number or value can vary by +/−20%.

The term “sterile” means that the noted device or material meets or exceeds defined medical guidelines of cleanliness and is substantially (if not totally) without contaminants so as to be suitable for medical uses.

Embodiments of the invention are particularly suitable for human or animal use.

The term “trocar” refers to a surgical instrument used to define an access path for laparascopic instruments into a patient body during a laparascopic surgical procedure. Trocars typically have a sharp leading end but may also have a blunt end. Trocars can be relatively slender or narrow for defining minimally invasive access paths to intrabody surgical sites. One or more trocars may be used during any particular surgery. The trocars may also be used with manual or robotic assisted surgical instruments. Exemplary trocars are available from a number of commercial suppliers including, for example, the Endopath® XCEL® trocars from Ethicon Endo-surgery, Inc. The trocars may have a channel diameter of between 3 mm to 20 mm, typically between 5-15 mm, but other sizes may be used.

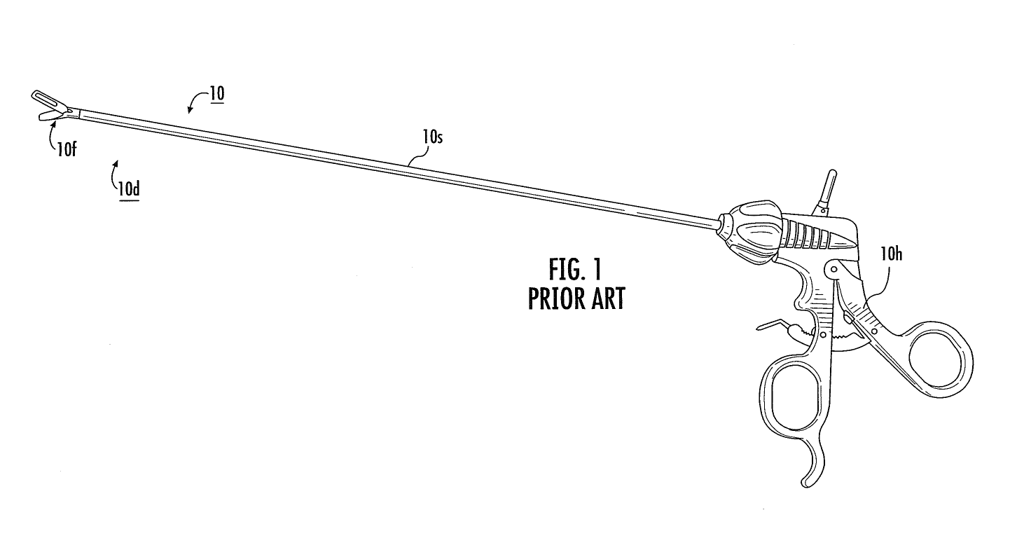

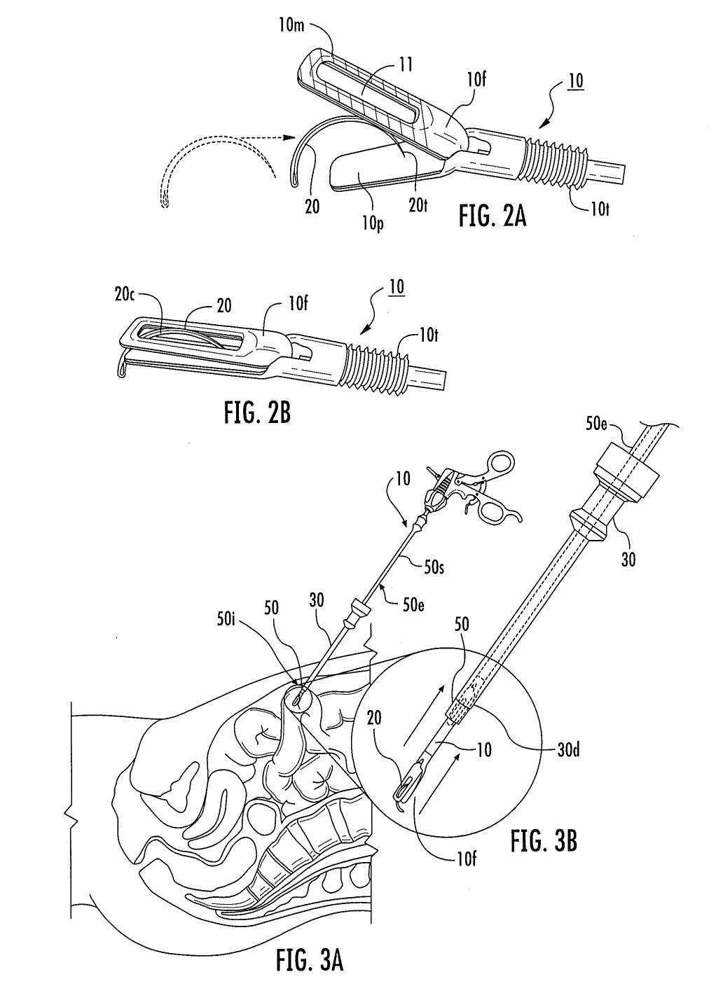

Turning now to the figures, FIG. 1 illustrates a conventional forceps 10 with a shaft 10s, handle 10h and forcep jaws 10f. The forcep jaws 10f are on a distal end of the shaft 10d. As shown in FIGS. 2A and 2B, the forcep jaws 10f may include threads 10t and can be threadably attachable to the shaft 10s. However, the jaws 10f can be formed integral to the shaft or may be attached using other assembly mechanisms.

As shown by the shading on the jaws in FIG. 2A, the jaws 10f can comprise a magnetic material 10m that can retrieve “lost” or dropped metallic needles. The jaws 10f can have a length between 0.5 inches to about 1.5 inches, but other sizes may be used.

FIGS. 2A and 2B also illustrate a suture needle 20 that can be grasped using the forcep jaws 101. Where the suture needle 20 is curved, as shown, one side of the jaws 10f can include a slot 11 through which a curved section of the needle 20c can protrude. The other jaw member can include a resilient padding 10p that accepts the sharp tip of the needle 20t. The forcep jaws 10f close together to trap the needle 20 therebetween as shown in FIG. 2B.

FIG. 3A illustrates an exemplary use with a trocar 30 that extends outside the body and a distance into the body to define an access path. FIG. 3B is an enlarged view of the device shown in FIG. 3B and more clearly shows the needle 20 grasped by the forcep jaws 10f. FIGS. 3A and 3B also show a needle lock 50 that is sized and configured to encase at least a portion of the forcep jaws 10f. The needle lock 50 can reside a distance outside the distal end of the trocar 30d as shown in FIG. 3A. The needle lock 50 can slidably engage the forcep jaws 10f to keep the jaws closed during retraction of the forceps 10 out of the trocar 30. The needle lock 50 can slidably advance a distance forward away from the distal end of the trocar 30d to encase or cover at least a portion of the force jaws 10f before the forceps jaws 10f are withdrawn into the trocar 30. Alternately, the lock 50 can remain stationary (or even retract) to slidably receive the forcep jaws 10f, as the forcep jaws 10f with the needle held therein is pulled or drawn toward the distal end of the trocar 30d.

The needle lock 50 can comprise a sleeve, a clip, band, ring or other component that can clamp the forcep jaws 10f shut with the needle 20 therebetween during retraction of the forceps with the needle out of the trocar 30. The needle lock 50 can comprise a sufficiently rigid polymeric and/or metallic material that can provide a suitable clamp force and can be sized and configured to fit inside the trocar 30 with the forceps 10. The needle lock 50 can abut the shaft of the forceps 10s and, when deployed, can snugly abut the forcep jaws so that a user cannot open the forcep jaws 10f when the needle lock 50 is in locking position.

The needle lock 50 can have a maxima cross-sectional dimension of between 2-15 mm, typically between about 5-15 mm. The needle lock 50 can have a maxima dimension that is within +10% of the size of the shaft 10s and/or jaws 10f.

FIG. 3A illustrates that the needle lock 50 can be a sleeve 50s. The sleeve 50s can have a length so that it can extend all the way through the trocar 30 with an external portion 50e and an internal portion 50i proximate the distal end of the trocar 30d. The sleeve 50s can have other lengths and may reside inside the trocar 30 rather than have an external portion when not in use (prior to deployment to withdraw the suture needle in the forceps). In some embodiments, the sleeve 50s can comprise a semi-rigid material that resists shape change when manually pressed by a user. The sleeve can be provided in different lengths for different end applications. The sleeve 50s can have a length of between about 30-50 cm, such as about 36 cm or about 45 cm. The sleeve 50s and cooperating forcep 10 can be provided as an assembly and sized for different uses. For example, for obese patients, a longer shaft and/or sleeve may be desired, such as about 40-50 cm.

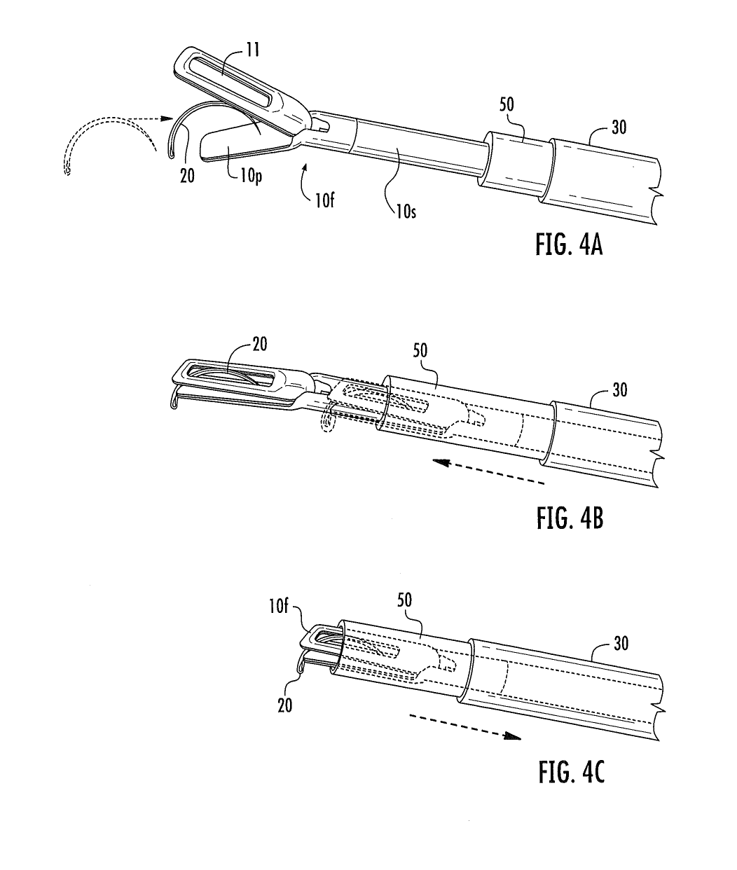

FIGS. 4A-4C illustrate an exemplary sequence that can be used to deploy the needle lock 50 and withdraw the locked forceps 10f with the needle 20 out of the trocar 30 according to some particular embodiments. Once the needle 20 is grasped by the forceps 10f, as shown in FIG. 4A, the needle lock 50 can be slidably advanced toward the grasped needle 20 as shown in FIG. 4B (and indicated by the arrow direction). The phantom illustration of the partially enclosed needle in FIG. 4B is an example of a target “lock configuration.” Once the needle lock 50 surrounds, encases or otherwise contacts at least a portion of the forcep jaws 10f, including a portion over the needle 20, the entire assembly is withdrawn together as a unit out of the trocar 30 (as indicated by the arrow direction).

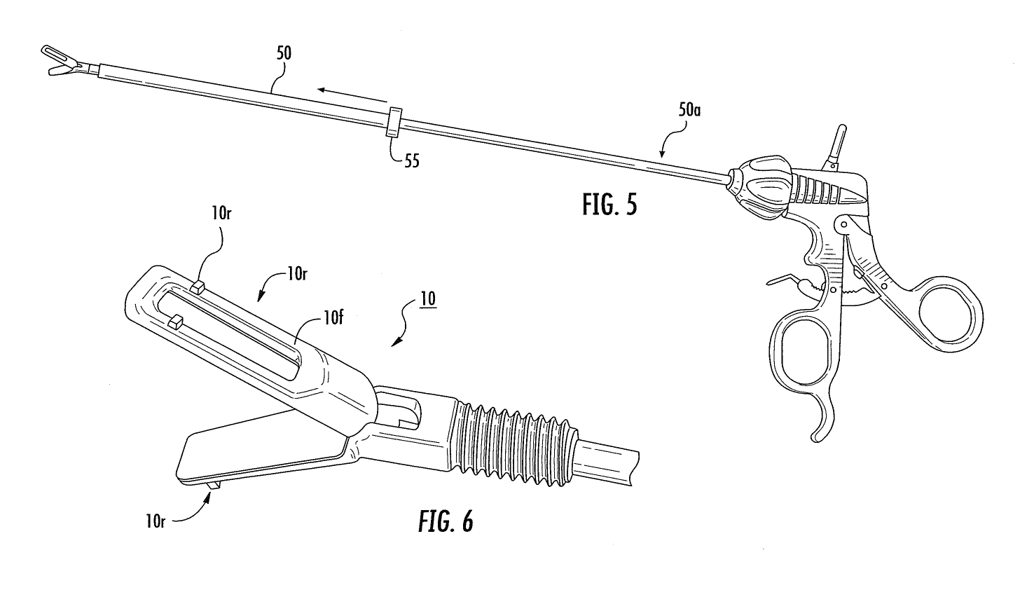

In some embodiments, as shown in FIG. 5, the surgical instrument assembly 50a can include an external locking member 55 that communicates with the needle lock 50 to prevent reverse sliding (in a direction away from the forcep jaws) once the needle lock 50 is in the operative lock position over the forceps jaw 10f, This locking member 55 can be a ratchet or a manually applied coupler that a user can clip or otherwise attach against the shaft 10s to secure the operative lock position of the needle lock 50 in place.

In some embodiments, as shown in FIG. 6, the forcep jaws 10f can include a position stop segment 10r proximate the distal end portion of the forcep jaws 10f to keep the needle lock 50 from sliding off the distal end when the lock 50 over the forceps 10 with the needle 20 is pulled back through the trocar 30. The position stop segment 10r can include a projecting member or members such as a ridge or ridges, studs or bumps, an increased friction surface finish such as an embossed surface and/or a supplemental resilient and/or compressible material to provide a cushion or padding to help stabilize the grasped needle tip. The padding or cushion can comprise a solid foam, polymer or rubber or other suitable, biocompatible (non-toxic) material. One or both of the forcep jaws 10f can include the stop segment 10r.

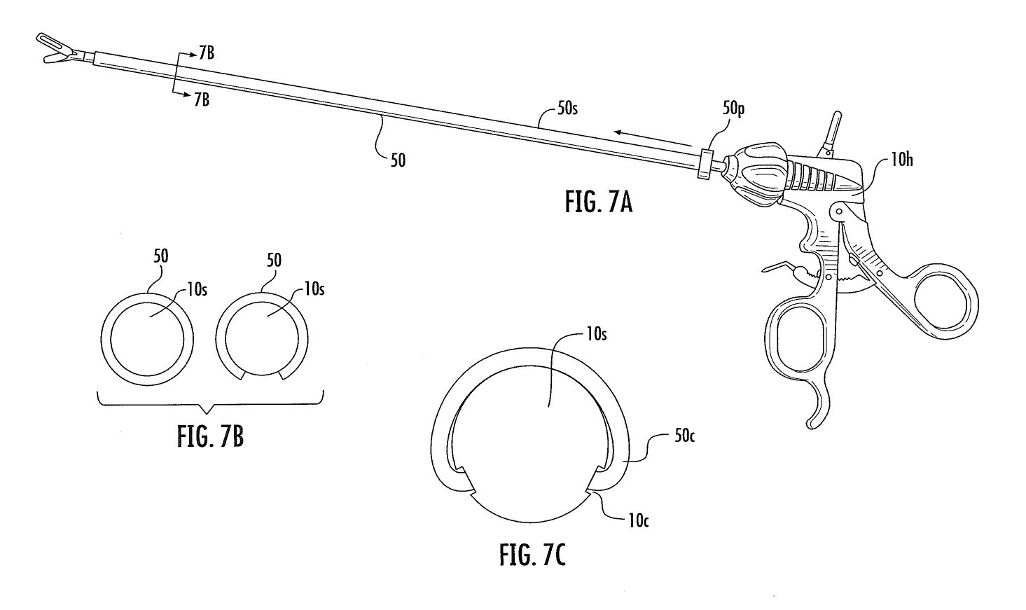

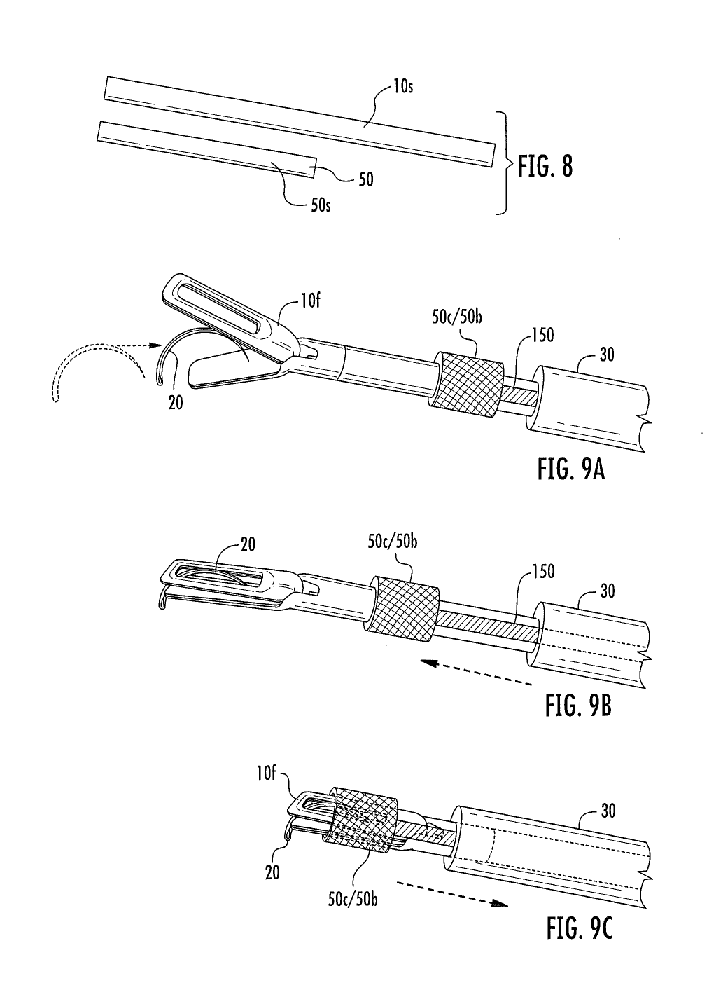

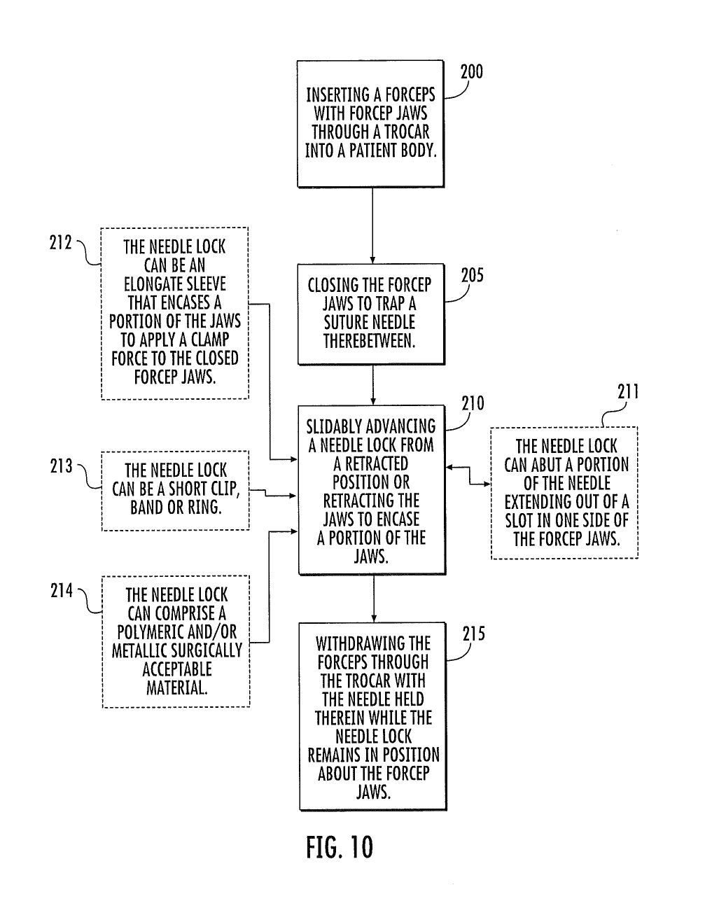

FIG. 7A illustrates that the needle lock 50 can include a grip portion 50p that can be used to slide the needle lock (or pusher 150, FIG. 9A) to deploy the needle lock 50. The grip portion 50p can be integral to the body of the needle lock or may be a separate component that attaches or moves against the needle lock to slidably deploy the needle lock into the lock position. The grip portion 50p can also define an anti-reverse lock member 55 as it can include vices, pinchers or clamps that can squeeze against the shaft 10s (over the needle lock sleeve or directly against the shaft 10s) to lock the needle lock 50 in the lock/deployed position. FIG. 7B illustrates that the needle lock 50 can have a perimeter, when viewed from the end, that is continuous, e.g., circular or discontinuous, e.g., a “C” shape.

FIG. 8 illustrates that the needle lock 50 can be a sleeve 50s that can be assembled to the shaft 10s of the forceps prior to attaching the threaded forcep jaws 10f. The shaft 10s can be at reduced temperature and/or the sleeve can be heated at assembly to facilitate a slidably snug fit of at least a distal end of the needle lock 50. The sleeve 50s can include different diameter portions.

As described above, the needle lock 50 can be configured in different manners. Although FIGS. 3A, 3B and 4A-4C show the use of a sleeve 50s as a tubular, cylindrical sleeve, the sleeve is not required to be cylindrical, but may have a “C” shape or discontinuous perimeter (with a longitudinally extending slit). Other embodiments include one or more C-shaped clips that can slide down the shaft of the forceps in a cooperating channel or tracks 10c that run the longitudinal axis (FIG. 7C)). Other embodiments include clips 50c shorter or rings/bands 50r that can be deployed via a pusher 150 as shown in FIGS. 9A-9C.

FIGS. 9A-9C illustrate that the needle lock 50 can include a clip 50c or band/ring 50b that can be deployed with a pusher 150. The pusher 150 can be a sleeve that is separate from the clip or band or may be attached thereto. The clip or band 50c/50b can be attached to the shaft or pusher with a tether to provide an affirmative physical accounting of the device and/or inhibit unwanted movement off of the forcep jaws. The pusher 150 can be a flat rigid or semi-rigid member that has sufficient rigidity to be able to push the clip or band onto the forcep jaws 10f. The pusher 150 can have a low profile so as to be able to fit inside the trocar with the forcep shaft 10s. The pusher can be arcuate (semi-circular) to conform to an outer surface of the shaft 10s or may reside in a groove in the shaft 10s (or even in a center channel (not shown)).

FIG. 10 is a flow chart of exemplary operations that can be used to carry out embodiments of the present invention. As shown, a forceps with forcep jaws is inserted through a trocar into a patient body (block 200). The forcep jaws are closed to trap a suture needle therebetween (block 205). A needle lock is slidably advanced from a retracted position to encase a portion of the jaws or the forcep jaws are retracted so that the needle lock encases at least a portion of the jaws (block 210). The forceps are withdrawn through the trocar with the needle held therebetween while the needle lock remains in position about the forceps jaws (block 215.

The needle lock can abut a portion of the needle extending out of a slot in one side of the forcep jaws (block 211).

The needle lock can be an elongate sleeve that encases at least a portion of the jaws to apply a clamp force to the closed forcep jaws (block 212).

The needle lock can be a short clip, band or ring (block 213).

The needle lock can comprise a polymeric and/or metallic surgically acceptable material (block 214).

While it is envisioned that the needle locks are particularly suitable for manual manipulation of the forceps, other embodiments contemplate use with robotic assisted minimally invasive surgeries.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

That which is claimed:

1. A laparoscopic surgical instrument, comprising:
forceps with a shaft attached to forcep jaws, the forceps being sized and configured to enter a trocar during a laparoscopic surgical procedure; and
a needle lock sized and configured to enter the trocar with the forceps and slidably receive at least a portion of the forcep jaws to clamp the forcep jaws shut while the forcep jaws hold a suture needle in a lock position therebetween.

2. The surgical instrument of claim 1, wherein the needle lock has a maxima cross-sectional size of between 2-15 mm.

3. The surgical instrument of claim 1, wherein the forcep jaws comprise cooperating first and second jaws, the first jaw having a slot therein and the second jaw having a padded surface thereon.

4. The surgical instrument of claim 1, wherein the forcep jaws comprise projecting members and/or a surface treatment or material that cooperates with the needle lock to define a stop position for the needle lock.

5. The surgical instrument of claim 1, wherein the needle lock comprises an elongate sleeve that abuts the shaft of the forceps.

6. The surgical instrument of claim 1, wherein the needle lock comprises a sleeve that is selectively slidably advanced toward the forcep jaws from a pre-deployment position proximate a distal end of the trocar.

7. The surgical instrument of claim 1, wherein the needle lock comprises a semi-rigid polymeric material sleeve and is sterile for medical use.

8. The surgical instrument of claim 1, wherein the needle lock comprises a clip that slidably resides about an external surface of the shaft of the forceps.

9. The surgical instrument of claim 1, wherein the needle lock comprises a ring or band that slidably resides about an external surface of the shaft of the forceps.

10. The surgical instrument of claim 1, further comprising a pusher that is in communication with the needle lock and abuts the shaft of the forceps when inside the trocar.

11. The surgical instrument of claim 5, further comprising an external grip accessible by a user that is attached to a proximal end portion of the sleeve.

12. The surgical instrument of claim 1, further comprising a locking member that attaches to the shaft of the forceps to prevent rearward sliding of the needle lock once the needle lock is in the lock position over the forcep jaws.

13. A method of withdrawing a suture needle during a laparoscopic surgery comprising:
inserting forceps with a forceps shaft and forcep jaws with a needle lock through a trocar;
grasping a suture needle with the forcep jaws; then
slidably receiving at least a portion of the forcep jaws with the grasped suture needle into a needle lock to clamp the jaws shut while holding the suture needle; and then
withdrawing the grasped suture needle from the trocar with the forcep jaws held inside the needle lock.

14. The method of claim 13, wherein the needle lock comprises a sleeve that extends out a distal end of the trocar.

15. The method of claim 14, wherein the slidably receiving step is carried out by slidably advancing the sleeve a distance outside the distal end of the trocar toward the forcep jaws.

16. The method of claim 13, wherein the needle lock comprises a clip or ring, and wherein the slidably receiving step is carried out by pushing the clip or ring a distance outside a distal end of the trocar toward the forcep jaws.
